(19) United States
(12) Patent Application Publication
(10) Pub. No.: US 2011/0230762 A1
(43) Pub. Date: Sep. 22, 2011

(54) COMPRESSION DEVICE USED IN ULTRASONIC MEASUREMENT, COMPRESSION CONTROL METHOD THEREOF, AND PHOTOACOUSTIC MEASUREMENT APPARATUS AND CONTROL METHOD THEREOF

(75) Inventors: Toshinobu Tokita, Yokohama-shi (JP); Katsumi Nakagawa, Yokohama-shi (JP)

(73) Assignee: Canon Kabushiki Kaisha, Tokyo (JP)

(21) Appl. No.: 13/131,942

(22) PCT Filed: Jan. 20, 2010
(86) PCT No.: PCT/JP2010/051000

§ 371 Date: May 31, 2011

(30) Foreign Application Priority Data
Jan. 21, 2009 (JP) 2009-010657

(51) Int. Cl. A61B 8/00; A61B 6/00

(52) U.S. Cl. 600/437; 600/407

(57) ABSTRACT

A compression device for compressing a subject that is used in ultrasonic measurement that receives ultrasonic waves from the subject to acquire biological information of the subject, comprising: two compression plates that are used when compressing the subject, and that face each other in a vertical direction with respect to a horizontal plane; a flexible sheet for setting a subject on, which is stretched between upper ends of the two compression plates; a compression mechanism that relatively changes a distance between the two compression plates to sandwich and compress the subject that is set on the flexible sheet through the flexible sheet; and a supply unit for supplying a matching liquid onto the flexible sheet. The apparatus can match an acoustic impedance with a subject and can suppress leaking or overflowing of a matching liquid.

### TECHNICAL FIELD

The present invention relates to a compression device used in ultrasonic measurement and a compression control method thereof, and a photoacoustic measurement apparatus and a control method thereof.

More particularly, the present invention relates to a compression device used in a photoacoustic measurement apparatus that radiates near infrared rays inside a subject, receives photoacoustic waves generated inside the subject with an ultrasound probe, and displays a tissue image inside the subject.

### BACKGROUND ART

Srirang Manohar, et al., The Twente photoacoustic mammoscope: system overview and performance, Physics in Medicine and Biology 50 (2005) 2543-2557 discusses a photoacoustic measurement apparatus that has been developed for use in breast cancer examination.

According to Manohar, et al., a subject (breast) is compressed between a glass plate and an ultrasound probe, and illumination light (near infrared rays) that employs an Nd:YAG laser as a light source is radiated to the breast through the glass plate.

Photoacoustic waves generated inside the subject (breast) are received with the ultrasound probe. The received photoacoustic waves are used to reconstruct an image of tissue inside the subject (breast), particularly angiogenesis in breast cancer, and the reconstructed image is displayed.

However, according to the apparatus discussed in Manohar, et al., gaps are caused due to dead spaces between a part of the subject (breast) and the ultrasound probe in which the part of subject (breast) cannot contact the ultrasound probe. Since the gap portions are filled with air and an acoustic impedance of air is significantly different from the acoustic impedance inside a subject, photoacoustic waves are not transmitted through the gap portions and regions arise in which image reconstruction cannot be performed.

Therefore, according to Japanese Patent Application Laid-Open No. S60-190853, a transmission-type ultrasonic measurement apparatus is proposed, by filling gap portions with an acoustic matching medium formed of water or a gel, the apparatus suppresses the occurrence of regions for which image reconstruction cannot be performed, to thereby match the acoustic impedances.

The ultrasonic measurement apparatus according to Japanese Patent Application Laid-Open No. S60-190853 will now be described using FIGS. 9A and 9B.

In FIGS. 9A and 9B, a compression device 901 compresses a breast as a subject 51. An accordion mechanism 905 is attached to the compression device 901.

An acoustic matching medium 906 for matching acoustic impedances is filled inside the compression device 901.

The ultrasonic measurement apparatus also includes a transmitting transducer 911a and a receiving transducer 911b.

The acoustic matching medium 906 is sealed by the compression device 901 and the accordion mechanism 905, and the compression device 901 is movable in a compressing direction.

However, there are the following problems with the conventional example as exemplified by ultrasonic measurement apparatus of Japanese Patent Application Laid-Open No. S60-190853.

Although according to the apparatus of Japanese Patent Application Laid-Open No. S60-190853, a problem as in the case of Manohar, et al. described above, in which the acoustic impedance differs because of gap portions and regions arise that cannot be measured, does not arise, another problem remains relating to retention of a matching liquid (acoustic matching medium 906).

That is, as illustrated in FIGS. 9A and 9B, when a form is adopted in which a subject is inserted from the side, when water is employed as a matching liquid (acoustic matching medium 906) there is a risk that the matching liquid (acoustic matching medium 906) will leak throughout between before and after the compressing by the compression device 901.

Alternatively, when a gel is employed as the matching liquid (acoustic matching medium 906), since the volume thereof changes by the compressing operation there is a risk that the matching liquid (acoustic matching medium 906) will overflow.

Further, in order to save time and labor for cleaning the compression device each time the person to be examined changes, it is necessary to make both a portion which the person to be examined contacts and the acoustic matching medium 906 disposable.

Furthermore, when inserting the subject into the acoustic matching medium 906, the subject should be inserted as deeply as possible. However, because the area surrounding the portion to be inserted is covered by the accordion mechanism 905 and the like, a large amount of time and trouble is required to insert the subject deeply.

Unless the subject can be inserted deeply into the compression device, regions that cannot be measured arise in proportion to the area of the subject that cannot be inserted into the acoustic matching medium 906. Therefore a unit to insert the subject as deeply as possible into the acoustic matching medium 906 is necessary.

### DISCLOSURE OF THE INVENTION

The present invention was made in view of the above problems, and an object of the invention is to provide, as a compression device for compressing a subject that is used in ultrasonic measurement that receives ultrasonic waves from the subject to acquire biological information of the subject, a compression device used in ultrasonic measurement that can match an acoustic impedance with a subject and can suppress leaking or overflowing of a matching liquid, as well as a compression control method thereof, and a photoacoustic measurement apparatus and a control method thereof.

Another object of present invention is to provide a compression device used in ultrasonic measurement that can make a portion contacted by a person to be examined as well as an acoustic matching medium disposable and can cause a subject to be inserted more deeply and decrease regions that is not measured, as well as a compression control method thereof, and a photoacoustic measurement apparatus and a control method thereof.

Therefore, the present invention is aimed to provide a compression device, a photoacoustic measurement apparatus, compression control methods and a control method of the photoacoustic measurement apparatus as below.

The compression device of the present invention for compressing a subject that is used in ultrasonic measurement that receives ultrasonic waves from the subject to acquire biological information of the subject, comprising: two compression plates that are used when compressing the subject, and that face each other in a vertical direction with respect to a horizontal plane; a flexible sheet for setting a subject on, which is stretched between upper ends of the two compression plates; a compression mechanism that relatively changes a distance between the two compression plates to sandwich and compress the subject that is set on the flexible sheet through the flexible sheet; and a supply unit for supplying a matching liquid onto the flexible sheet.

The photoacoustic measurement apparatus of the present invention that comprises a compression device which sandwiches and compresses a subject between two compression plates, and that compresses a subject using the compression device and detects as a photoacoustic signal an ultrasonic wave generated by a light irradiated at the subject, characterized in that the compression device is a compression unit comprising the compression device as above; the photoacoustic measurement apparatus further comprising: an illumination light optical system for irradiating the light at a subject through the compression plate; a probe that receives a photoacoustic signal generated from the subject through the compression plate; and a processing portion that processes a photoacoustic signal received by the probe to reconstruct an image.

The compression control method of the present invention by a compression device for compressing a subject that is used in ultrasonic measurement that receives ultrasonic waves from the subject to acquire biological information of the subject, comprising: stretching a flexible sheet between upper ends of two compression plates that face each other in a vertical direction with respect to a horizontal plane; setting a subject between the facing compression plates from a top surface of the flexible sheet, and detecting completion of the setting; starting compressing driving by a compression mechanism in order to compress the subject by means of the two compression plates; ending compressing driving by the compression mechanism; and filling a matching liquid between the flexible sheet and the subject.

The other compression control method of the present invention by a compression device for compressing a subject that is used in ultrasonic measurement that receives ultrasonic waves from the subject to acquire biological information of the subject, comprising: stretching a flexible sheet between upper ends of two compression plates that face each other in a vertical direction with respect to a horizontal plane; forming a hollow for supplying a matching liquid to and accommodating a subject on in a top surface of the flexible sheet; setting the subject in the hollow for accommodating the subject, and detecting that setting of the subject is completed; starting compressing driving by a compression mechanism in order to compress the subject by means of the two compression plates; ending compressing driving by the compression mechanism; and filling a matching liquid between the flexible sheet and the subject.

The control method of the present invention for a photoacoustic measurement apparatus that comprises a compression device which sandwiches and compresses a subject between two compression plates, and that compresses a subject using the compression device and detects as a photoacoustic signal an ultrasonic wave generated by light that is made incident on the subject, comprising: compressing a subject using the compression device as above; receiving through a probe a photoacoustic signal that is propagated from the subject through at least one of the compression plates when light is irradiated at the subject through at least one of the compression plates among the two compression plates; and processing a photoacoustic signal received by the probe and reconstructing an image.

According to the present invention it is possible to realize, as a compression device for compressing a subject that is used in ultrasonic measurement that receives ultrasonic waves from the subject to acquire biological information of the subject, a compression device used in ultrasonic measurement that can match an acoustic impedance with a subject and can suppress leaking or overflowing of a matching liquid, as well as a compression control method thereof, and a photoacoustic measurement apparatus and a control method thereof.

Further, it is possible to realize a compression device used in ultrasonic measurement that can make a portion contacted by a person to be examined as well as an acoustic matching medium disposable and can cause a subject to be inserted more deeply and decrease regions that can not be measured, as well as a compression control method thereof, and a photoacoustic measurement apparatus and a control method thereof.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a view that describes a configuration of a compression device according to Embodiment 1 of the present invention;

FIG. 2 is a flowchart of a process from setting a subject inside a compressing container until compressing the subject according to Embodiment 1 of the present invention;

FIGS. 3A, 3B, 3C and 3D are views that describe a process from setting a subject inside a compressing container until compressing the subject according to Embodiment 1 of the present invention, in which FIG. 3A is a view that describes a step of detecting a subject has been set, and FIGS. 3B to 3D are views that describe a step of filling a matching liquid;

FIG. 4 is a view that describes the configuration of a compression device according to Embodiment 2 of the present invention;

FIG. 5 is a flowchart of a process from setting a subject inside a compressing container until compressing the subject according to Embodiment 2 of the present invention;

FIG. 6 is a view that describes sheet replenishment according to Embodiment 2 of the present invention;

FIG. 7 is a view that describes the configuration of a compression device according to Embodiment 3 of the present invention;

FIG. 8 is a view that describes a photoacoustic apparatus on which a compression device is mounted according to Embodiment 4 of the present invention; and

FIGS. 9A and 9B are views that describe the background art according to a conventional example.

### BEST MODE FOR CARRYING OUT THE INVENTION

Preferred embodiments of the present invention will now be described in detail in accordance with the accompanying drawings.

Embodiments of the present invention are described hereunder.

A compression device of the present embodiments is a compression device for compressing a subject that is used in ultrasonic measurement that receives ultrasonic waves from a subject to acquire biological information of the subject. The compression device is configured as follows.

More specifically, in order that the device can match an acoustic impedance with that of a subject, and insert and compress a subject while also suppressing leakage or overflowing of a matching liquid, the device is arranged as follows.

The compression device includes two compression plates that face each other in a vertical direction with respect to a horizontal plane, and are used when compressing the subject; and a flexible sheet for setting a subject on that is stretched between upper ends of the two compression plates that face each other in the vertical direction.

The compression device also includes a compression mechanism that relatively changes a distance between the two compression plates facing each other in the vertical direction so as to sandwich and compress the subject that is set on the flexible sheet through the flexible sheet; and a supply unit for supplying a matching liquid onto the flexible sheet. According to this configuration, the subject can be set on a flexible sheet from the top surface, leakage or overflowing of a matching liquid for matching acoustic impedances from the compression device can be reduced, and the subject can be compressed.

By providing the flexible sheet with a hollow for accommodating the subject, it is possible to facilitate setting of the subject when setting the subject on the flexible sheet. Further, by arranging the flexible sheet so as to be detachable with respect to the upper end of the compression plates, a portion which a person to be examined contacts and an acoustic matching medium can be made disposable. By adopting a configuration that has an actuator that pulls one end of the flexible sheet downward while sandwiching the subject, the subject can be set deeply in the compression device, and hence regions that cannot be measured can be reduced.

The compression device of the present invention is used in ultrasonic measurement that receives ultrasonic waves from a subject to acquire biological information of the subject, and for example is mounted to the following type of apparatus. The apparatus is a photoacoustic measurement apparatus that radiates pulsed light at a subject and receives ultrasonic waves emitted due to a local temperature increase that occurs when the pulsed light is absorbed. This is referred to as so-called PAT (Photo Acoustic Tomography), and ultrasonic waves generated from a subject are also referred to as photoacoustic waves. Since light is attenuated significantly inside a living body, a compression device such as that of the present invention can be suitably applied for performing photoacoustic measurement for a deep portion of a subject. The compression device of the present invention can also be applied to a known ultrasound apparatus that radiates ultrasonic waves at a subject and receives ultrasonic waves (reflection echo) reflected from the subject.

In the present specification, the term “matching liquid” refers to a material that is supplied between a probe that receives ultrasonic waves and a subject, and has an acoustic impedance value that is approximate to that of the subject. The concept of “matching liquid” also includes a gel and the like as long as the material has no fixed shape and cannot be retained without a structure such as that of the present invention.

### EMBODIMENTS

Hereunder, embodiments of the present invention are described.

### Embodiment 1

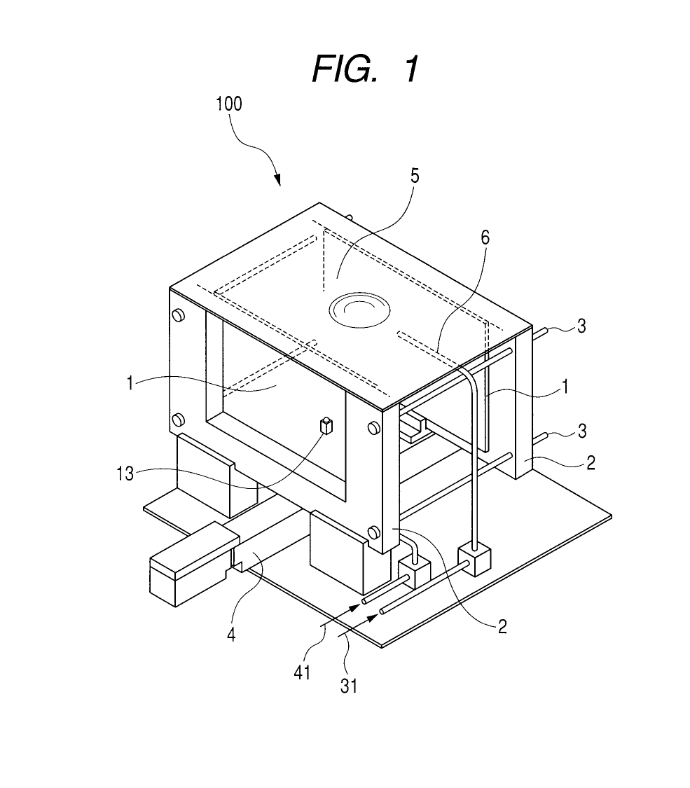

According to Embodiment 1, a configuration example of a compression device that applies the present invention is described. FIG. 1 illustrates a view that describes the configuration of a compression device according to the present embodiment.

The configuration illustrated in FIG. 1 includes compression plates 1, compression plate fastening portions 2, guides 3, a compression mechanism 4, and a sheet for setting a subject on 5. Arrows 31 and 41 indicate directions in which matching liquid and lubricant are introduced into the apparatus, respectively.

The configuration illustrated in FIG. 1 also includes a matching liquid supply line 6, a lubricant supply nozzle 13, and a compression device 100.

In the compression device 100 of this embodiment, the compression plates 1 for sandwiching a subject are composed of two flat plates that face each other in a vertical direction with respect to a horizontal plane. The two flat plates can be composed of parallel flat plates.

The compression plates 1 are retained by the compression plate fastening portions 2, respectively, and the parallelism between the compression plates 1 is maintained by the guides 3.

The compression plates 1 are driven by the compression mechanism 4 so that the compression plates 1 are made to relatively approach each other or move away from each other.

The guides 3 and the compression mechanism 4 can be mounted to the compression plate fastening portions 2.

Although according to the present embodiment the compression mechanism 4 is composed by a robot mechanism, the compression mechanism 4 is not limited thereto, and may be an air cylinder mechanism or a manual mechanism (vise mechanism) that uses a rack-and-pinion or a worm gear.

In the compression device 100 of the present embodiment, the sheet 5 for setting a subject on is stretched between the upper ends of two flat plates that face each other in the vertical direction as described above. At this time, it is desirable that the sheet 5 for setting a subject on is formed with a flexible sheet, and is formed in a shape that is provided with a concave portion (hollow) in a central part that facilitates setting of the subject thereon. Regarding the material of the sheet 5, a flexible sheet formed with a rubber material such as urethane rubber or latex rubber is suitable.

A rubber material is suitable because the acoustic impedance of a rubber material is close to that of the subject and thus facilitates matching of the respective acoustic impedances. Furthermore, the sheet 5 can change shape and therefore facilitates setting of a subject thereon regardless of the size of the subject.

The material of the sheet 5 is not limited to a rubber material, and a resin film such as polyethylene or PET or a vinyl are also available.

However, when using a resin film or a vinyl for the sheet 5, creases can easily occur when compressing the subject with the compression plates 1.

Therefore, because creases hinder matching of acoustic impedances, sheets 5 can be prepared in advance that include a plurality of types of concave portion sizes that conform to the sizes of subjects.

Furthermore, in consideration of hygiene, the sheet 5 can be adapted to be removable with respect to the upper ends of the compression plates, and can be replaced each time the person under examination changes.

Therefore, fixing portions (unshown) for fixing the sheet 5 can be provided, for example, at the upper parts of the compression plate fastening portions 2, and the sheet 5 is replaced for each person under examination.

Liquid for matching the acoustic impedance between the sheet 5 and the subject that is set thereon is filled by a matching liquid supply line 6.

As the liquid for matching the acoustic impedance, deaerated water, gel or oil is suitable.

The matching liquid supply line 6 includes the following members in addition to pipe. For example, the matching liquid supply line 6 includes a pump for feeding a matching liquid, an adjustment valve for adjusting a flow rate or a pressure when feeding liquid, a heater for adjusting a temperature of the matching liquid to a temperature near that of the subject, a filter that eliminates foreign substances from the matching liquid, and a valve that controls opening and closing of liquid feeding.

Further, matching liquid (lubricant) for matching acoustic impedances can also be supplied between the surfaces of the two compression plates 1 facing each other and a surface that the subject does not contact of the flexible sheet.

Thus, even if a crease occurs in the sheet 5, matching liquid enters the crease and the acoustic impedances are matched.

Furthermore, the matching liquid serves as a lubricant and enables compressing of the subject while suppressing the occurrence of creases.

An application method using an atomizer or a scraper is suitable with respect to a method for supplying the matching liquid (lubricant), and both automated application and manual application can be used.

As illustrated in FIG. 1, according to the present embodiment a lubricant supply nozzle 13 that utilizes an atomization method is provided as a supply unit for supplying a lubricant.

Water or castor oil as a lubricant is applied between the compression plates 1 and the sheet 5 using the lubricant supply nozzle 13.

A lubricant may also be applied with an unshown scraper. In that case, gel is also effective as a lubricant.

The compression device 100 of the present embodiment has the above described configuration. However, there is a possibility that the lifetime of the machine will be shortened due to the matching liquid leaking and wetting the compression mechanism 4 as a result of the sheet 5 breaking or a hole forming therein.

Therefore, a pan (unshown) can be provided below the sheet 5 as a precaution in case the matching liquid leaks.

Next, the process of a compression control method from a time that a subject is set inside a compressing container until compressing begins using the compression device of the present embodiment will be described.

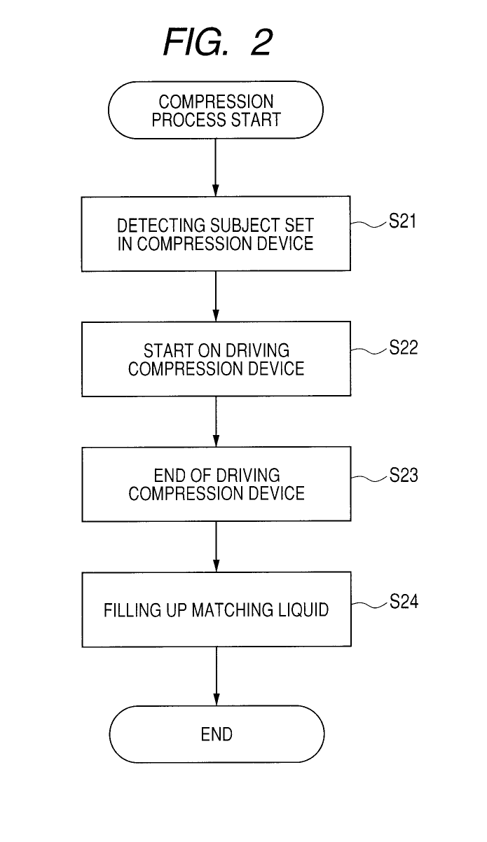

FIG. 2 is a flowchart of a process from setting a subject inside a compressing container until compressing the subject according to the present embodiment.

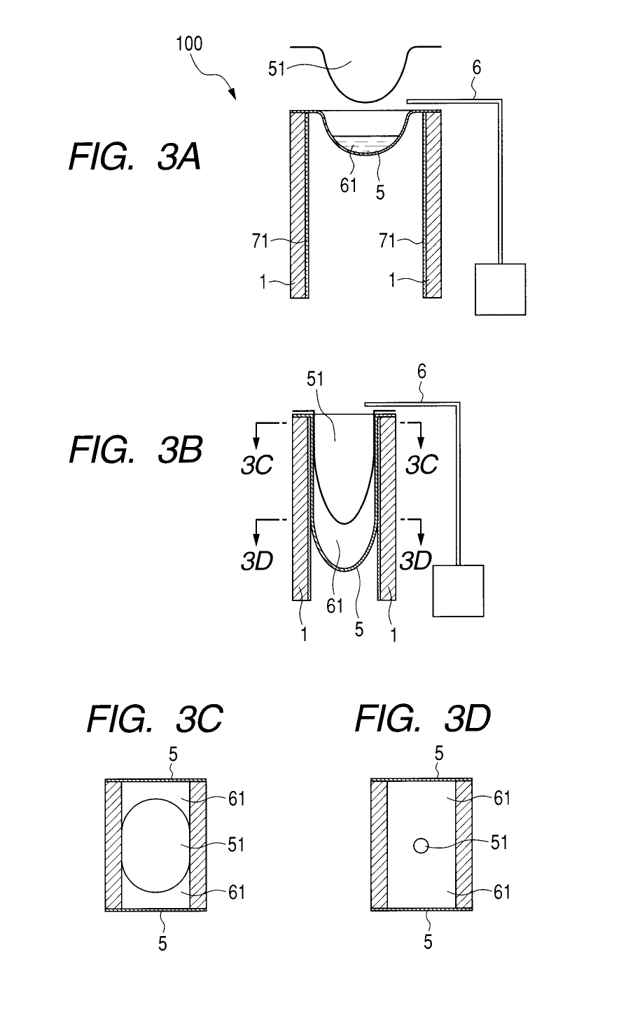

FIGS. 3A to 3D are views that describe a process from setting a subject inside a compressing container until compressing the subject according to the present embodiment, in which FIG. 3A is a view that describes a step of detecting that a subject has been set, and FIGS. 3B to 3D are views that describe a step of filling matching liquid.

As shown in FIG. 2, first, in a step S21 of detecting that a subject has been set, a subject is set in a concave portion of the sheet 5 and setting of the subject is detected.

At that time, a small amount of matching liquid can be desirably supplied to inside the concave portion of the sheet 5 from the matching liquid supply line 6.

FIG. 3A is a view that illustrates the above described state from a side surface direction of the compression plates 1.

A detection unit is used to detect that setting of a subject 51 on the above described flexible sheet is completed. And the detection unit may take the form of operation of an unshown button that is intended as a setting detection after a technician such as a physician or an operator sets a subject in the concave portion of the sheet 5.

Other available detection methods include attaching a strain gauge to the sheet 5, measuring deformation of the sheet 5 after a subject is set thereon, and detecting that setting is completed when the deformation amount is saturated.

Next, in step S22 in which the compression mechanism starts compressing driving, the subject is compressed by the compression mechanism 4.

Subsequently, in step S23 in which compressing driving by the compression mechanism ends, the compression mechanism 4 ends the driving when a distance between the compression plates 1 becomes a predetermined value. Alternatively, a sensor that measures a compressing force is provided and the compression mechanism 4 ends the driving when an output of the sensor becomes a predetermined value.

Next, in step S24 of filling a matching liquid, the concave portion of the sheet 5 is filled with a matching liquid 61 from the matching liquid supply line 6.

FIGS. 3B to 3D are views that illustrate the above described state from the side surface direction of the compression plates 1.

As is clear from the state in S24 as illustrated in FIGS. 3B to 3D, the acoustic impedance of a subject that is compressed by the compression plates 1 is matched as far as the compression plates via the matching liquid and the sheet 5.

According to the above process, it is possible to deform the sheet 5 and fill a matching liquid between the sheet 5 and the subject.

Therefore, the acoustic impedance can be matched from the subject to the compression plates 1.

Further, if the sheet 5 is made disposable, time and labor for cleaning can be saved and the usability can be improved.

### Embodiment 2

In Embodiment 2, a configuration example of a compression device which is provided with a sheet that has a flat central part, and that does not have a concave portion in a central part as in Embodiment 1, is described.

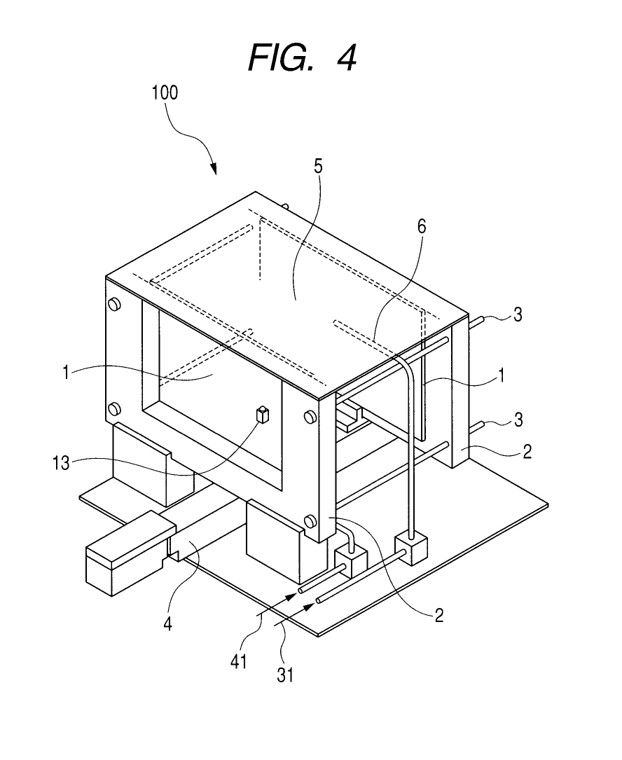

FIG. 4 is a view that describes the configuration of a compression device according to the present embodiment.

Components in FIG. 4 which are the same as in FIG. 1 that illustrates Embodiment 1 are denoted by the same reference numerals, and a description of those common components is omitted hereunder.

According to the present embodiment, the sheet 5 has a flat central part and is stretched between the upper ends of two flat plates that face each other in the vertical direction.

Because costs may increase due to processing of the sheet 5 when a concave portion is provided in the central part of the sheet 5 as in Embodiment 1, according to the present embodiment a sheet 5 in which the central part is also flat is used.

Next, a process from a time that a subject is set in a compressing container until compressing begins using the compression device of the present embodiment as illustrated in FIG. 4 will be described.

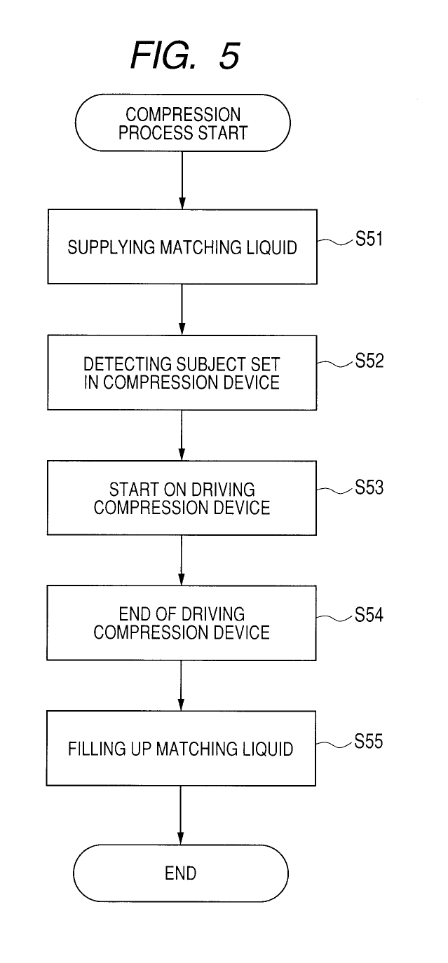

FIG. 5 is a flowchart of a process from setting a subject inside a compressing container until compressing the subject according to the present embodiment.

As shown in FIG. 5, first, in step S51 of supplying a matching liquid in, a matching liquid is supplied to the sheet 5 from the matching liquid supply line 6.

As a result, the sheet 5 bends in a concave shape under the weight of the matching liquid. More specifically, the sheet 5 becomes approximately the same shape as the sheet 5 that has a concave portion as described in Embodiment 1.

Next, in step S52 of detecting that a subject has been set, a subject is set in the sheet 5 and it is detected that setting of the subject is completed.

Next, in step S53 in which the compression mechanism starts compressing driving, the subject is compressed by the compression mechanism 4.

Subsequently, in step S54 in which compressing driving by the compression mechanism ends, the compression mechanism 4 ends the driving when a distance between the compression plates 1 becomes a predetermined value. Alternatively, a sensor that measures a compressing force is provided and the compression mechanism 4 ends the driving when an output of the sensor becomes a predetermined value.

Next, in step S55 of filling a matching liquid, a matching liquid is filled into the concave portion of the sheet 5 from the matching liquid supply line 6.

Thus, according to Embodiment 2, processing that is performed in advance to provide a concave portion in the central part of the sheet 5 need not be performed.

Further, a sheet roller 7 described below can be provided as a unit that stretches the sheet in detachable condition between the upper ends of the two flat plates facing each other in the vertical direction.

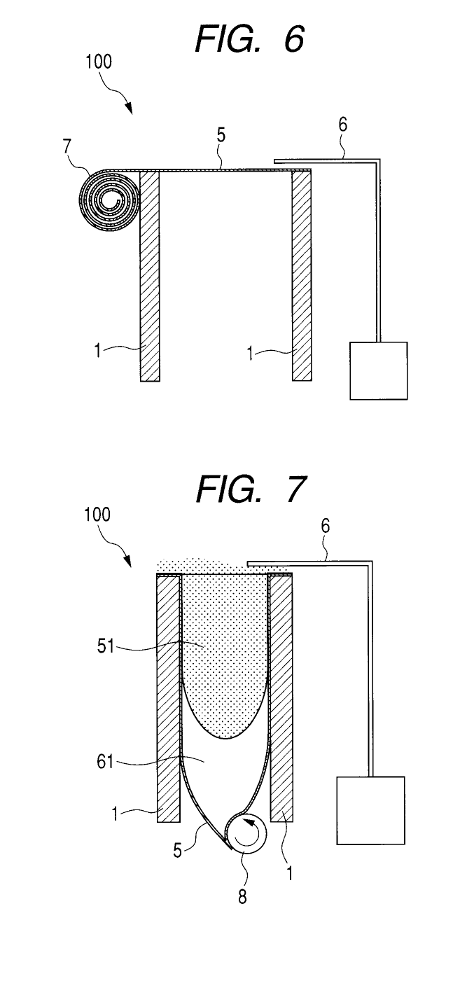

For example, for the sheet roller 7, as illustrated in FIG. 6, a configuration may be adopted in which the sheet 5 is provided on a roller, and each time a person under examination changes the sheet 5 is peeled off and a new sheet 5 is pulled out from the sheet roller 7 and fixed.

As a result, a portion that a person to be examined contacts and an acoustic matching medium can be made disposable, and thus the usability can be enhanced to an even greater degree.

### Embodiment 3

In embodiment 3, a configuration example of a compression device that compresses a subject more deeply into the compression device is described.

FIG. 7 is a view that describes the configuration of a compression device according to the present embodiment.

Components in FIG. 7 that are the same as in FIG. 1 that illustrates Embodiment 1 are denoted by the same reference numerals, and a description of those common components is omitted hereunder.

In the compression device of the present embodiment, an actuator that pulls one end of a sheet downward while sandwiching a subject is provided below two flat plates that face each other in the vertical direction.

The above described Embodiment 1 and Embodiment 2 are each configured such that a subject is set on the sheet 5 and compressed together with the sheet 5 between the compression plates 1 by the compression mechanism 4. However, in the present embodiment, as described above, an actuator is provided for compressing a subject more deeply into a compression device.

According to the present embodiment, the actuator is composed by a roller 8 that rolls up the sheet 5.

When the sheet 5 is rolled up by the roller 8, the sheet 5 is pulled to the roller side. Accompanying such pulling of the sheet 5, the subject is also pulled in the downward direction in FIG. 7. Thus, the subject can be compressed more deeply into the compression device.

A step of rolling up the sheet 5 using the roller 8 may preferably be performed when the subject is being compressed, and may preferably be performed between the step in which driving of the compression mechanism starts in S22 and the step in which driving of the compression mechanism ends in S23 as illustrated in FIG. 2 of Embodiment 1.

Alternatively, a step of rolling up the sheet 5 using the roller 8 may also preferably be performed between the step in which driving of the compression mechanism starts in S53 and the step in which driving of the compression mechanism ends in S54 as illustrated in FIG. 5 of Embodiment 2.

A matching liquid 71 for matching acoustic impedances can be applied between the compression plates 1 and the sheet 5.

Thus, by means of an action as a lubricant of the matching liquid, sliding of the sheet 5 with respect to the compression plates 1 improves (frictional resistance decreases) and the subject can be compressed more deeply into the compression device.

An application method using an atomizer or a scraper is suitable with respect to a unit that supplies the matching liquid (lubricant) 71, and both automated application and manual application can be used.

Further, the step of rolling up the sheet 5 using the roller 8 can be performed in the step of supplying a matching liquid in S51 as illustrated in FIG. 5 of Embodiment 2.

Since the step S51 in FIG. 5 is aimed to form a concave portion for setting a subject on in the sheet 5, an arrangement may also be adopted in which the sheet 5 is rolled up with the roller 8 at that time to facilitate setting of the subject.

Thus far, a description has been made regarding a method that rolls up the sheet 5 with the roller 8 in order to pull the sheet 5 downward. However, a method of pulling the sheet 5 downward is not limited thereto, and may be another method as long as the method is one that pulls one portion of the sheet 5 downward. For example, a method may be adopted in which a portion of the sheet 5 is hooked by a member that is rectilinearly moved by an actuator, and the member is rectilinearly moved to pull the sheet 5 downward.

Thus, according to the present embodiment a subject can be set more deeply into a compression plate 1.

Therefore, by combining this feature with matching of acoustic impedances, regions which can not be measured can be reduced further.

### Embodiment 4

In Embodiment 4 a configuration example of a photoacoustic measurement apparatus to which the compression device of Embodiment 1 is mounted is described. The photoacoustic measurement apparatus radiates near infrared rays to inside a subject, receives photoacoustic signals that are generated inside the subject and propagate therefrom with an ultrasound probe, and displays a tissue image of inside the subject.

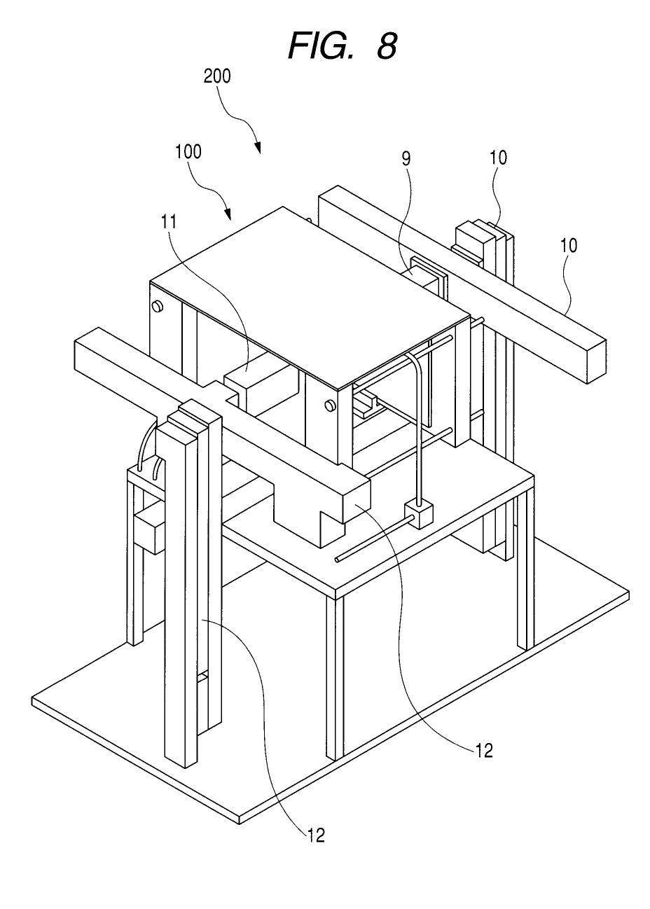

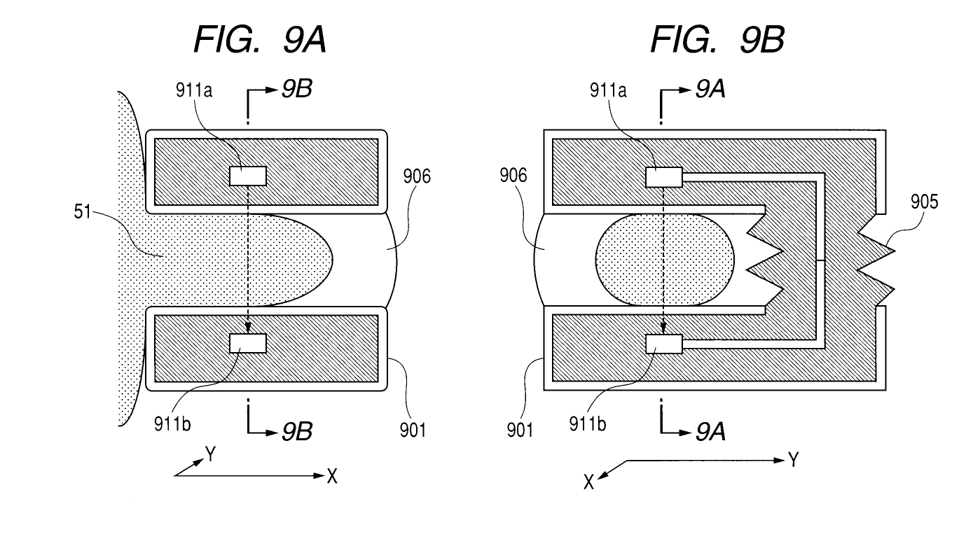

FIG. 8 illustrates the configuration of a photoacoustic measurement apparatus (photoacoustic mammography, hereunder referred to as “PAM”).

The term “photoacoustic waves” refers to acoustic waves that are generated when near infrared rays radiated to inside a subject are absorbed by tissues inside the subject and undergo local thermal expansion.

By receiving the photoacoustic waves with an ultrasound probe and extracting (detecting) a signal unique to the photoacoustic waves, the tissue of the subject that absorbed the near infrared rays can be specifically imaged.

By radiating a wavelength that is easily absorbed by blood (hemoglobin) in particular among the subject tissues, an image of blood or a blood vessel can be specifically acquired, and in particular angiogenesis at a cancer cite can be imaged.

As a result, the degree of progression of a cancer can be ascertained in more detail.

FIG. 8 illustrates an apparatus in which the principles of this type of photoacoustic measurement apparatus that radiates near infrared rays to inside a subject to cause the rays to be incident thereon receives photoacoustic signals generated inside the subject with an ultrasound probe, and displays a tissue image of inside the subject are applied to breast cancer screening.

In FIG. 8, the compression device described using FIG. 1 to FIG. 7 is denoted by reference numeral 100.

An illumination light optical system 9 is a system for irradiating laser beams of a wavelength from 650 nm to 1100 nm for generating photoacoustic waves from a subject (breast).

The illumination light optical system can be composed by an illumination light optical system for irradiating near infrared rays at a subject through at least one flat plate (through a compression plate) of the parallel flat plates.

In this connection, a laser light source and a route of illumination light from the laser light source to the illumination light optical system 8 are not shown in the drawing.

An illumination light scan unit 10 scans near infrared rays from the illumination light optical system and adjusts an irradiation position.

A probe 11 receives photoacoustic waves emitted from a subject (breast). A probe scan unit 12 is used for scanning the probe 11 and aligning the probe inside a predetermined region.

In this case, the compression plate 1 on the side on which the probe is arranged can have favorable transmissivity with respect to photoacoustic waves, and preferably made of a resin, particularly, polymethylpentene as a material thereof.

Further, the compression plate 1 on the side of the illumination light optical system 9 can have favorable transmissivity with respect to illumination light, and preferably made of a resin such as polycarbonate or glass as a material thereof.

According to the photoacoustic measurement apparatus of the present embodiment, photoacoustic signals received by the probe 11 are amplified, subjected to A/D conversion, and undergo processing such as delay and sum, and envelope detection, and the resulting signals are used to perform three-dimensional image reconstruction.

The image information obtained by the three-dimensional image reconstruction processing is displayed on a monitor.

The relevant processing portions and the monitor are not illustrated in FIG. 8. Known methods may be applied as the methods for performing signal processing of photoacoustic signals to performing reconstruction of a three-dimensional image, and the present invention is not limited to a method described herein.

It should be understood that the range of application of the compression device 100 of the present invention is not limited to the PAM apparatus 200. For example, eliminating the illumination light optical system 9 and the illumination light scan unit 10 and using a probe 11 that can send and receive ultrasonic waves enables application of the compression device 100 an ultrasound apparatus.

By mounting the compression device 100 described above to a photoacoustic apparatus, time and labor required for cleaning can be reduced. Hence, the operating efficiency of the photoacoustic apparatus can be improved.

The compression device and the control method thereof of the present invention described in the foregoing can also be applied to an ultrasound apparatus, and not only a photoacoustic apparatus.

The present invention is not limited to the above embodiments and various changes and modifications can be made within the spirit and scope of the present invention. Therefore to apprise the public of the scope of the present invention, the following claims are made.

This application claims the benefit of Japanese Patent Application No. 2009-010657, filed Jan. 21, 2009, which is hereby incorporated by reference herein in its entirety.

1. A compression device for compressing a subject that is used in ultrasonic measurement that receives ultrasonic waves from the subject to acquire biological information of the subject, comprising:
two compression plates that are used when compressing the subject, and that face each other in a vertical direction with respect to a horizontal plane;
a flexible sheet for setting a subject on, which is stretched between upper ends of the two compression plates;
a compression mechanism that relatively changes a distance between the two compression plates to sandwich and compress the subject that is set on the flexible sheet through the flexible sheet; and
a supply unit for supplying a matching liquid onto the flexible sheet.

2. The compression device according to claim 1, wherein a hollow for accommodating the subject is provided in the flexible sheet.

3. The compression device according to claim 1, wherein the flexible sheet is arranged so as to be detachable with respect to the upper ends of the compression plates.

4. The compression device according to claim 1, further comprising a detection unit that detects that the subject is set on the flexible sheet.

5. The compression device according to claim 1, including further comprising a supply unit that supplies a lubricant between surfaces at which the two compression plates face each other and a surface of the flexible sheet that the subject does not contact.

6. The compression device according to claim 1, further comprising an actuator that pulls one end of the flexible sheet downward while sandwiching the subject.

7. A photoacoustic measurement apparatus that comprises a compression device which sandwiches and compresses a subject between two compression plates, and that compresses a subject using the compression device and detects as a photoacoustic signal an ultrasonic wave generated by a light irradiated at the subject,
wherein the compression device is a compression unit comprising the compression device according to claim 1, and
the photoacoustic measurement apparatus further comprising:
an illumination light optical system for irradiating the light at a subject through the compression plate;
a probe that receives a photoacoustic signal generated from the subject through the compression plate; and
a processing portion that processes a photoacoustic signal received by the probe to reconstruct an image.

8. The photoacoustic measurement apparatus according to claim 7, wherein, of the two compression plates, a compression plate on a side on which the probe is disposed is formed by resin.

9. The photoacoustic measurement apparatus according to claim 8, wherein the resin comprises polymethylpentene.

10. A compression control method of a compression device for compressing a subject that is used in ultrasonic measurement that receives ultrasonic waves from the subject to acquire biological information of the subject, comprising the steps of:
stretching a flexible sheet between upper ends of two compression plates that face each other in a vertical direction with respect to a horizontal plane;
setting a subject between the facing compression plates from a top surface of the flexible sheet, and detecting completion of the setting;
starting compressing driving by a compression mechanism in order to compress the subject by means of the two compression plates;
ending compressing driving by the compression mechanism; and
filling a matching liquid between the flexible sheet and the subject.

11. A compression control method of a compression device for compressing a subject that is used in ultrasonic measurement that receives ultrasonic waves from the subject to acquire biological information of the subject, comprising the steps of:
stretching a flexible sheet between upper ends of two compression plates that face each other in a vertical direction with respect to a horizontal plane;
forming a hollow for supplying a matching liquid to and accommodating a subject on in a top surface of the flexible sheet;
setting the subject in the hollow for accommodating the subject, and detecting that setting of the subject is completed;
starting compressing driving by a compression mechanism in order to compress the subject by means of the two compression plates;
ending compressing driving by the compression mechanism; and
filling a matching liquid between the flexible sheet and the subject.

12. The compression control method of a compression device according to claim 10,
the method further comprising the step of, after detecting that setting of the subject is completed, supplying a lubricant to between surfaces at which the two compression plates face each other and a surface of the flexible sheet that the subject does not contact.

13. The compression control method of a compression device according to claim 10,
the method further comprising the step of, between starting compressing driving by the compression mechanism and ending compressing driving by the compression mechanism, pulling one end of the flexible sheet downward by means of an actuator to cause a subject to be set more deeply in the compression device.

14. A control method of a photoacoustic measurement apparatus that comprises a compression device which sandwiches and compresses a subject between two compression plates, and that compresses a subject using the compression device and detects as a photoacoustic signal an ultrasonic wave generated by light that is made incident on the subject, comprising the steps of:
pressing a subject using a compression device according to of claim 1;
receiving through a probe a photoacoustic signal that is propagated from the subject through at least one of the compression plates when light is irradiated at the subject through at least one of the compression plates among the two compression plates; and
processing a photoacoustic signal received by the probe and reconstructing an image.
